(19) United States
(12) Patent Application Publication
(10) Pub. No.: US 2011/0165305 A1
(43) Pub. Date: Jul. 7, 2011

(54) Method for preparing a protein hydrolysate

(75) Inventors: Gitte Budolfsen Lynglev, Frederiksberg (DK); Peter Rahbek Oestergaard, Virum (DK); Lars Lehmann Hilling Christensen, Alleroed (DK); Tine Hoff, Holte (DK); Daniel U. Staerk, Kirkwood, MO (US); Theodore M. Wong, Ballwin, MO (US); Phillip s. Kerr, Wildwood, MO (US)

(73) Assignees: Novozymes A/S, Bagsvaerd (DK); Solae LLC, St. Louis, MO (US)

(21) Appl. No.: 13/054,527

(22) PCT Filed: Jul. 17, 2009
(86) PCT No.: PCT/US2009/050999

§ 371 Date: Mar. 16, 2011

Related U.S. Application Data
(60) Provisional application No. 61/081,780, filed on Jul. 18, 2008.

(51) Int. Cl. A23C 9/00; A23L 1/10; A23L 1/31; A23J 1/00; C12P 21/06; C07K 14/00

(52) U.S. Cl. 426/580; 426/619; 426/641; 426/656; 435/68.1; 530/350

(57) ABSTRACT

The present invention relates to a method for preparing a protein hydrolysate. The invention further relates to a protein hydrolysate produced using the method, and to food products comprising such protein hydrolysate.

### REFERENCE TO SEQUENCE LISTING

This application contains a sequence listing in computer readable form. The computer readable form is incorporated herein by reference.

### FIELD OF THE INVENTION

The present invention relates to a method for preparing a protein hydrolysate. The invention further relates to a protein hydrolysate produced using the method, and to food products comprising such protein hydrolysate.

### BACKGROUND OF THE INVENTION

Edible products comprising added protein or protein compositions are known to be desired as food or food supplements to humans and animals. Such food supplements are known to confer beneficial effects on health and physical appearance for the consumers. Protein materials for food products are often hydrolysed. It is desired to have a certain degree of hydrolysis in certain food product, as increasing the degree of hydrolysis has beneficial effects in terms of functionality of the protein material (i.e. an increased degree of hydrolysis can improve solubility and suspendability and may reduce the viscosity and color of the protein material). Especially when used in beverages the degree of hydrolysis is of importance. However, food products containing hydrolysed protein materials are often found by consumers to be less desired as the hydrolysation often confers a bitter taste to the product. Especially industrially produced hydrolysed proteins, e.g. derived from milk, yeast or soy, may be less tasty than desired by consumers. The cause of the distaste of some of these protein materials is known to be a sensation of bitterness.

U.S. Pat. No. 6,372,282 relates to a process for obtaining protein hydrolysates using an exopeptidase in combination with one or more endoproteases to improve process economics.

EP 1512328 A1 describes the use of a fungal protease mixture having both endo- and exopeptidase activity for the hydrolysis of soy protein.

WO 2001/30172 A1 describes a method for producing cheese or cheese-derived products comprising use of a Kluveromyces lactis cell or an enzyme derived therefrom. Among many other enzymes is mentioned use of carboxypeptidase Y from Kluveromyces lactis.

EP 323930 describes use of a carboxypeptidase to eliminate bitterness of peptides obtained by hydrolysis of proteinaceous matter.

### SUMMARY OF THE INVENTION

During the course of the experiments leading to the present invention it was surprisingly shown that the use of a carboxypeptidase Y was superior in reducing the bitterness of protein hydrolysates and food products, compared to conventional enzymatic treatment processes.

Accordingly, one aspect of the present invention relates to a process for preparing a protein hydrolysate from a protein containing material comprising the steps of
a. hydrolyzing the protein containing material,
b. contacting the hydrolysed protein material with a carboxypeptidase comprising an amino acid sequence which has at least 70% identity to the amino acid sequence of SEQ ID NO:1 or amino acids 124-542 thereof.

In one particular aspect, the invention relates to a product obtainable by use of a process according to the invention.

Other aspects and features of the invention are described in more detail below.

### DEFINITIONS

SEQ ID NO: 1 shows the amino acid sequence of CPY from Aspergillus oryzae as determined from the DNA sequence of its gene. SEQ ID NO: 2 shows the amino acid sequence of the mature enzyme as determined from the N-terminal sequence. SEQ ID NO: 3 shows the N-terminal sequence as determined experimentally.

The terms “peptidase” or “protease” both refer to an enzyme that hydrolyses peptide bonds in oligopeptide or polypeptide chains. The group of peptidases comprises the enzymes assigned to subclass EC 3.4.

The terms “endoprotease” or “endopeptidase” both refer to an enzyme that hydrolyses internal peptide bonds in oligopeptide or polypeptide chains. The group of endoproteases comprises the enzymes assigned to subclasses EC 3.4.21-25.

The term “endoprotease, which preferentially produces C-terminal hydrophobic amino acid residues” are defined herein as an endoprotease which has preference for cleavage of internal peptide bonds in peptides, polypeptides or proteins at the C-terminal side of hydrophobic amino acid residues. Defined in a general manner, the endoprotease activity is directed towards internal hydrophobic amino acid residues, thus substantially producing peptide fragments with hydrophobic amino acid residues at the C-terminals of the peptide fragments. By “endoprotease, which preferentially produces C-terminal hydrophobic amino acid residues” is not meant that the protease may not have other activities, but only that the major activity is towards cleaving internal hydrophobic amino acid residues at the carboxy terminal side.

The term “carboxypeptidase” refers to an enzyme that cleaves the C-terminal peptide bond of a peptide or polypeptide chain. The group comprises the enzymes assigned to enzyme subclass EC 3.4.16-18. Carboxypeptidases release a single amino acid residue from the carboxy terminal end.

The term “carboxypeptidase C” refers to an enzyme that cleaves the C-terminal peptide bond of a peptide or polypeptide chain with a preference for hydrophobic residues in positions P1 and P1′. The group comprises the enzymes assigned to enzyme subclass EC 3.4.16.5. A particularly preferred subclass of Carboxypeptidase C is Carboxypeptidase Y.

A “hydrolysate” is a reaction product obtained when a compound is cleaved through the effect of water. Protein hydrolysates occur subsequent to thermal, chemical, or enzymatic degradation. During the reaction, large protein molecules are broken into smaller proteins, soluble proteins, peptide fragments, and free amino acids.

The terms “endoprotease activity” or “hydrolysing endoprotease activity” are defined herein as a protease activity which catalyzes the cleavage of internal peptide bonds in peptides, polypeptides or proteins.

The term “carboxypeptidase activity” is defined herein as a peptidase activity which catalyzes the cleavage of the carboxy-terminal peptide bond in peptides, polypeptides or proteins.

The term “hydrophobic amino acid residue” is defined herein as belonging to the group of residues of the amino acids tryptophan, phenylalanine, tyrosine, histidine, leucine, isoleucine, valine, cysteine, methionine, proline and alanine. The hydrophobic character of an amino acid residue can be ordered based on the change in Gibbs free energy for hydrophobic association (Urry, D. W., “The change in Gibbs free energy for hydrophobic association; Derivation and evaluation by means of inverse temperature transitions.”, Chemical Physical Letters 399, 177-183, (2004)), which gives the following hydrophobicity scale for amino acid residues at pH 7:

| Amino acid | | ΔG°_{HA} |
|---|---|---|
| residue | Abbreviation | (kcal/mol) |
| Trp | W | −7.00 |
| Phe | F | −6.15 |
| Tyr | Y | −5.85 |
| His | H⁰ | −4.80 |
| Leu | L | −4.05 |
| Ile | I | −3.65 |
| Val | V | −2.50 |
| Cys | C | −1.90 |
| Met | M | −1.50 |
| Pro | P | −1.10 |
| Ala | A | −0.75 |
| Thr | T | −0.60 |
| Asn | N | −0.05 |
| Gly | G | −0.00 |
| Ser | S | +0.55 |
| Gln | Q | +0.75 |
| Arg | R⁺ | +0.80 |
| Lys | K⁺ | +2.94 |
| Asp | D⁻ | +3.4 |
| Glu | E⁻ | +3.72 |

“Food product” as defined herein is meant to include all protein-containing products intended for human consumption and/or clinical nutrition. In the broadest sense food products comprise solid food products as well as fluid food products. Food products include but is not limited to instant powder products, food bars, anti-fatigue protein hydrolysates, meat and meat analogue products, breakfast cereals, dairy products, soy milk products, infant formula, clinical nutrition products, baby food, extrudates (e.g., pasta), yeast hydrolysates, vegetable hydrolysates (e.g., soy hydrolysates, pea protein hydrolysates, canola protein hydrolysates), and meat/fish hydrolysates.

“Extrudate” as used herein refers to any food product formed by extrusion or using extruded dough, such as nuggets, crisps, puffs, and pasta.

The “degree of hydrolysis” (DH) expresses the extent of protein hydrolysis. In the context of the invention, the degree of hydrolysis (DH) is defined as follows: DH=(Number of peptide bonds cleaved/Total number of peptide bonds)×100%. The skilled person will know how to measure the DH.

### DETAILED DESCRIPTION OF THE INVENTION

During the course of the experiments leading to the present invention it was shown that carboxypeptidase Y (CPY) from Aspergillus oryzae was capable of reducing the bitterness of protein compositions and food products. This peptidase belongs to the group of peptidases known as carboxypeptidase C. Carboxypeptidase C enzymes have carboxypeptidase activity towards carboxy-terminal hydrophobic amino acid residues. All enzymes belonging to this class of peptidases are carboxypeptidases preferentially targeting hydrophobic amino acid residues.

Accordingly, one aspect of the present invention relates to the use of a carboxypeptidase enzyme for preparing a hydrolysate from a protein material.

More specifically, one aspect of the present invention provides for the use of peptidases having carboxypeptidase activity, which peptidases comprise an amino acid sequence which has at least 70% identity to the amino acid sequence of SEQ ID NO:1 or amino acids 124-542 thereof, for preparing a protein hydrolysate.

In a preferred aspect the present invention provides for the use of peptidases having carboxypeptidase activity, which peptidases comprise an amino acid sequence which has at least 71%, such as at least 72%, 73%, 74%, 75%, 76%, 77%, 78%, 79% or 80% identity to the amino acid sequence of SEQ ID NO:1 or amino acids 124-542 thereof, for preparing a protein hydrolysate. More specifically, the present invention provides for the use of enzymes or peptidases having carboxypeptidase activity, which peptidases comprise an amino acid sequence which has at least at least 81%, such as at least 82%, 83%, 84%, 85%, 86%, 87%, 88%, 89% or 90% identity to the amino acid sequence of SEQ ID NO:1 or amino acids 124-542 thereof, for preparing a protein hydrolysate. More specifically, the present invention provides for the use of enzymes or peptidases having carboxypeptidase activity, which peptidases comprise an amino acid sequence which has at least at least 91%, such as at least 92%, 93%, 94%, 95%, 96%, 97%, 98%, 99% or 100% identity to the amino acid sequence of SEQ ID NO:1 or amino acids 124-542 thereof, for preparing a protein hydrolysate.

In a preferred aspect the carboxypeptidase enzymes used consist of an amino acid sequence of a mature enzyme, or a fragment thereof. In a particular aspect the amino acid identities mentioned above are based on amino acids 124-542 of SEQ ID NO: 1.

The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

In one aspect of the invention the carboxypeptidase used is a carboxypeptidase C. In one aspect the carboxypeptidase C according to the invention is an enzyme classified as EC 3.4.16.5 in the NC-IUBMB classification. In one aspect of the invention the carboxypeptidase used is a carboxypeptidase Y.

In one aspect of the invention the carboxypeptidase is obtained from a fungus belonging to the genus Aspergillus. In one aspect of the invention the carboxypeptidase is a carboxypeptidase from Aspergillus oryzae.

Protein containing materials may be hydrolysed by several means known in the art e.g. mechanically, chemically or enzymatically. However, the present inventors further found that the use of a carboxypeptidase as described above, in combination with a hydrolysing endoprotease, was superior in reducing the bitterness while obtaining a hydrolysate having a desired degree of hydrolysis.

Accordingly, in one aspect the invention relates to the use of a carboxypeptidase in combination with a hydrolyzing endoprotease for the preparation of a hydrolyzed protein material. It is preferred that the carboxypeptidase has the sequence characteristics as described above.

In one particular aspect, the endoprotease is an endoprotease, which preferentially produces C-terminal hydrophobic amino acid residues in the hydrolysed protein material as defined above. In another aspect the endoprotease is an endoprotease which produces a protein hydrolysate that is more bitter than the starting protein material. In one aspect of the invention the endoprotease is a subtilisin (e.g. Savinase, Esperase, Alcalase). Preferred are subtilisins classified by NC-IUBMB classification EC 3.4.21.62. In one aspect of the invention the endoprotease is Alcalase or Savinase. In one aspect of the invention the endoprotease is a serine protease or a metalloprotease (like Neutrase). In one aspect the endoprotease is a serine protease from Nocardiopsis, such as a serine protease from Nocardiopsis prasina.

Endoproteases found to be applicable according to the present invention are e.g., the protease derived from Nocardiopsis sp. NRRL 18262 disclosed in WO88/03947 (here the strain is referred to as Nocardiopsis sp. strain 10R) and WO01/58276. Other related endoproteases which are useful according to the invention are disclosed in WO88/03947, WO04/111220, WO04/111222, WO04/111223, WO05/123911, and WO04/072279. As other examples of bacterial endoproteases applicable for use according to the invention can be mentioned the protease from Nocardiopsis alba (previously Nocardiopsis dassonvillei) NRRL 18133 disclosed in WO88/03947, the endoproteases from Nocardiopsis dassonvillei subsp. dassonvillei DSM 43235, Nocardiopsis alba DSM 15647, Nocardiopsis sp. DSM 16424 and the synthetic Protease 22, all four disclosed in WO04/111220, the protease from Nocardiopsis prasina DSM 15648 disclosed in WO04/111222, the protease from Nocardiopsis prasina DSM 15649 disclosed in WO04/111223, the proteases from Nocardiopsis prasina (previously Nocardiopsis alba) DSM 14010, Nocardiopsis alkaliphila DSM 44657 and Nocardiopsis lucentensis DSM 44048, all three disclosed in WO05/123911, the proteases from Brachysporiella gayana CGMCC 0865, Metarhizium anisopliae, Gliocladium sp. CBS 114001, Periconia sp. CBS 114002, Periconia sp. CBS 114000 and Curvularia lunata CBS 114003, all 6 disclosed in WO04/072279, and mutants, variants or fragments of any of these exhibiting endoprotease activity.

In another aspect the endoprotease is an acidic endoprotease, e.g. an acidic aspartic acid endoprotease, such as chymosin or rennilase.

As shown during the course of the present invention, and as illustrated in the examples, digestion of proteins or hydrolyzed proteins with enzyme preparations comprising carboxypeptidases that have carboxypeptidase activity towards hydrophobic amino acid residues (i.e. cleaves the protein material on the amino terminal side of a C-terminal hydrophobic amino acid residue), results in compositions comprising polypeptide fragments having improved characteristics in terms of reduced bitterness. Accordingly, such preparations are useful for production of food products having a reduced bitterness.

In one preferred aspect, the invention is directed at the use of a carboxypeptidase for reducing the bitterness of a protein hydrolysate.

Preferred uses of a carboxypeptidase according to the present invention can be summarized as the following preferred embodiments of the invention:

1. Use of a carboxypeptidase for preparing a protein hydrolysate from a protein containing material, wherein the carboxypeptidase comprises an amino acid sequence which has at least 70% identity to (i) the amino acid sequence of SEQ ID NO:1 or (ii) amino acids 124-542 thereof.
2. Use according to embodiment 1, where the carboxypeptidase comprises an amino acid sequence which has at least 80% identity to the amino acid sequence of SEQ ID NO:1 or amino acids 124-542 thereof.
3. Use according to embodiment 2, where the carboxypeptidase comprises an amino acid sequence which is identical to the amino acid sequence of SEQ ID NO:1 or amino acids 124-542 thereof.
4. Use according to embodiment 3, where the carboxypeptidase is the carboxypeptidase of SEQ ID NO:1 or amino acids 124-542 thereof.
5. Use according to any of the preceding embodiments, wherein the carboxypeptidase is a carboxypeptidase C.
6. Use according to any of the preceding embodiments, wherein the carboxypeptidase is a carboxypeptidase Y.
7. Use according to any of the preceding embodiments, wherein the protein containing material is hydrolysed by an endoprotease.
8. Use according to embodiment 7, where the endoprotease is an endoprotease which preferentially produces a hydrophobic amino acid in the C-terminal of the hydrolysed protein material.
9. Use according to embodiment 7 or 8, wherein the endoprotease is an endoprotease which produces a protein material that is more bitter than the starting protein material.
10. Use according to any of the preceding embodiments for reducing the bitterness of a protein hydrolysate.
11. Use according to any of the preceding embodiments, wherein the protein containing material is selected from the group consisting of animal protein containing materials, vegetable protein containing materials, yeast protein containing materials or derivatives and combinations thereof.
12. Use according to any of the preceding embodiments, wherein the protein containing material is selected from the group consisting of milk protein containing materials, soy protein containing materials, yeast protein containing materials or derivatives and combinations thereof.
13. Use according to any of the preceding embodiments, wherein the protein containing material is a soy protein containing material.
14. Use according to any of the preceding embodiments, wherein the carboxypeptidase is obtained from a fungus belonging to the genus Aspergillus.
15. Use according to embodiment 14, wherein the carboxypeptidase is obtained from Aspergillus oryzae.

### Process for Preparing a Protein Hydrolysate

In one aspect, the present invention provides a process for preparing a protein hydrolysate, the process comprising contacting hydrolysed protein with a carboxypeptidase enzyme. Thereby, the bitter taste of the protein material is reduced or removed.

The present inventors found that specific carboxypeptidases, namely the carboxypeptidases comprising an amino acid sequence which has at least 70% identity to the amino acid sequence of SEQ ID NO:1 or amino acids 124-542 thereof, are especially suited for removing the bitter taste of hydrolysed protein materials. Accordingly, in a particular aspect, the present invention provides a process for preparing a protein hydrolysate, the process comprising contacting hydrolysed protein with a carboxypeptidase comprising an amino acid sequence which has at least 70% identity to the amino acid sequence of SEQ ID NO:1 or amino acids 124-542 thereof.

In a preferred aspect, the process comprises contacting hydrolyzed protein with a carboxypeptidase comprising an amino acid sequence which has at least 71%, such as at least 72%, 73%, 74%, 75%, 76%, 77%, 78%, 79% or 80% identity to the amino acid sequence of SEQ ID NO:1 or amino acids 124-542 thereof. More specifically, it is preferred that the process comprises contacting hydrolyzed protein with a carboxypeptidase comprising an amino acid sequence which has at least 81%, such as at least 82%, 83%, 84%, 85%, 86%, 87%, 88%, 89% or 90% identity to the amino acid sequence of SEQ ID NO:1 or amino acids 124-542 thereof. More specifically, it is preferred that the process comprises contacting hydrolyzed protein with a carboxypeptidase comprising an amino acid sequence which has at least 91%, such as at least 92%, 93%, 94%, 95%, 96%, 97%, 98%, 99% or 100% identity to the amino acid sequence of SEQ ID NO:1 or amino acids 124-542 thereof.

Hydrolysed protein may be obtained by any method known in the art, e.g. chemical, mechanical (physical), or enzymatic hydrolysis. Enzymatic hydrolysis is, however, preferred, potentially conferring the advantage of simultaneous hydrolysis and treatment with the carboxypeptidase according to the invention. The treatment steps according to the invention may however also be performed sequentially (e.g. in order to optimize the process parameters of the enzymatic reactions to each enzyme). In a preferred aspect the enzyme used for hydrolyzing the protein material is an endoprotease.

Accordingly, the process may further comprise contacting the protein material with an endoprotease, particularly an endoprotease that specifically cleaves the protein material on the carboxy terminal side of internal hydrophobic amino acid residues prior to or simultaneous with contacting the resulting material with the carboxypeptidase according to the invention. Thereby, the protein material may be hydrolyzed to reduce the size and bitterness within a single process.

Accordingly, in one aspect the invention relates to a process for preparing a protein hydrolysate comprising contacting hydrolyzed protein with a carboxypeptidase C and an endoprotease, preferably an endoprotease that specifically cleaves the protein material on the carboxy terminal side of internal hydrophobic amino acid residues.

The process according to the invention is preferably a process for reducing the bitterness of a protein hydrolysate. In one aspect, the process according to the invention is a process for reducing the bitterness of a food product.

In the process according to this aspect of the invention, the protein material is typically mixed or dispersed in water to form a slurry comprising about 1% to about 20% protein by weight. In one aspect, the slurry may comprise about 1% to about 5% protein by weight. In another aspect, the slurry may comprise about 6% to about 10% protein by weight. In a further aspect, the slurry may comprise about 11% to about 15% protein by weight. In still another aspect, the slurry may comprise about 16% to about 20% protein by weight.

After the protein material is dispersed in water, the slurry of protein material may be heated from about 70° C. to about 90° C. for about 2 minutes to about 20 minutes to inactivate putative endogenous protease inhibitors and to denature or partly denature the protein material thereby making the substrate more accessible to the enzymes.

Typically, the pH and the temperature of the protein slurry are adjusted so as to optimize the enzymatic hydrolysis reaction and, in particular, to ensure that the carboxypeptidase and the optional hydrolyzing endoprotease used in the reaction function near their optimal activity level. If the carboxypeptidase and the optional endoprotease have different pH and/or temperature optima, the hydrolysis with the endoprotease may be carried out at a pH and temperature suitable for the endoprotease, and afterwards pH and/or temperature is adjusted to a level which is optimal for the carboxypeptidase reaction.

The pH of the protein slurry may be adjusted and monitored according to methods generally known in the art.

For the carboxypeptidase reaction, the pH of the protein slurry or the hydrolyzed protein slurry may be adjusted and maintained at from about 4.0 to about 8.0. In one aspect, the pH of the protein slurry or the hydrolyzed protein slurry may be adjusted and maintained at from about 5.0 to about 7.0. In another aspect, the pH of the protein slurry or the hydrolyzed protein slurry may be adjusted and maintained at from about 5.5 to about 6.5. In a preferred aspect, the pH of the protein slurry or the hydrolyzed protein slurry may be adjusted and maintained at about 6.0.

The temperature of the protein slurry or the hydrolyzed protein slurry is preferably adjusted and maintained at from about 25° C. to about 65° C. before and/or during the reaction with the carboxypeptidase in accordance with methods known in the art. In a preferred aspect, the temperature of the protein slurry or the hydrolyzed protein slurry may be adjusted and maintained at from about 40° C. to about 60° C. before and/or during the reaction with the carboxypeptidase. In general, temperatures above this range may inactivate the carboxypeptidase, while temperatures below this range tend to slow the activity of the carboxypeptidase. In a preferred aspect, the temperature of the protein slurry or the hydrolyzed protein slurry may be adjusted and maintained at from about 45° C. to about 55° C. before and/or during the reaction with the carboxypeptidase.

When optimal conditions are attained, the protein material is contacted with the peptidase having carboxypeptidase activity according to the invention and reacted for the desired length of time in order to obtain the desired degree of carboxypeptidase hydrolysis. In a particular aspect, the reaction is continued for 15 to 300 minutes, preferably for between 30 and 240 minutes, and more preferably for between 45 and 120 minutes.

In a preferred aspect, the hydrolysis reaction is initiated by adding a hydrolyzing endoprotease having endoprotease activity to the slurry of protein material. Several endoproteases are suitable for use in the process of the invention. Typically, the optimal conditions in terms of pH and temperature are well known to the person skilled in the art. In one aspect the endoprotease may be an enzyme having optimal activity at a pH from about 6.0 to about 11.0 and at a temperature from about 40° C. to about 65° C. In another aspect the endoprotease is an acidic endoprotease, e.g. an acidic aspartic acid endoprotease, which when used according to the invention allows for hydrolysis at a lower pH, e.g. pH 2-4.

The amount of carboxypeptidase used or added to the protein material according to the invention can and will vary depending upon the desired degree of removal of C-terminal hydrophobic residues and the duration of the hydrolysis reaction. The amount may range from about 1 mg to about 1000 mg of carboxypeptidase per kilogram of protein. In another aspect, the amount may range from about 10 mg to about 1000 mg of carboxypeptidase per kilogram of protein. In yet another aspect, the amount may range from about 10 mg to about 200 mg per kilogram of protein material.

The amount of hydrolyzing endoprotease optionally added to the protein material can and will vary depending upon the desired degree of hydrolysis and the duration of the hydrolysis reaction. The amount may range from about 1 mg to about 1500 mg of endoprotease per kilogram of protein. In another aspect, the amount may range from about 50 mg to about 1000 mg of endoprotease per kilogram of protein. In yet another aspect, the amount may range from about 250 mg to about 550 mg per kilogram of protein material.

As will be appreciated by a skilled artisan, the duration of the process steps according to the invention can and will vary. Generally speaking, the duration of the hydrolysis reaction may range from a few minutes to several hours, such as, from about 30 minutes to about 24 hours.

Optionally, after or in concurrence with the hydrolyzing endoprotease digestion and the treatment with one or more carboxypeptidases according to the present invention, the hydrolysate composition may be contacted with one or more additional exopeptidases, such as carboxypeptidases, or one or more additional endoproteases.

To end the hydrolysis reaction, the composition may be heated to a temperature that is high enough to inactivate the enzymes used. For example, heating the composition to a temperature of approximately 90° C. will substantially heat inactivate the endoproteases.

### Protein Material

The protein material to be processed according to the invention is selected from the group consisting of animal protein containing materials, vegetable protein containing materials, yeast protein containing materials or derivatives and combinations thereof. Preferably, the protein material is selected from the group consisting of milk protein material, soy protein material, yeast protein material or derivatives and combinations thereof.

In one aspect of the invention the protein material is yeast protein material.

In one aspect of the invention the protein material is milk protein material.

In a preferred aspect of the invention the protein material is soy protein material.

In a preferred aspect, the protein material to be treated with a carboxypeptidase according to the invention is a soy protein material, such as a hydrolysed soy protein material, or a soy protein hydrolysate.

A hydrolysed protein material to be treated with a carboxypeptidase according to the invention preferably has a degree of hydrolysis of from about 0% to about 30%. More preferably the degree of hydrolysis vary from 5%-20%.

### Soy Protein Material

A variety of soy protein materials may be used in the process of the invention to generate soy protein hydrolysates. In general, the soy protein material is derived from whole soybeans in accordance with methods known in the art. The whole soybeans may be standard soybeans (i.e., non genetically modified soybeans), commoditized soybeans, genetically modified soybeans, and combinations thereof. In an exemplary embodiment, the soybean is hybridized GMO.

In one aspect, the starting material used in the process may be a soy protein isolate (also called isolated soy protein, or ISP). In general, soy protein isolates have a protein content of at least about 90% soy protein on a moisture-free basis. The soy protein isolate may comprise intact soy proteins or it may comprise partially hydrolysed soy proteins. Examples of soy protein isolates that are useful in the present invention are commercially available, for example, from Solae, LLC (St. Louis, Mo.), and include SUPRO® 500E, SUPRO® 620, and SUPRO® EX 33.

In another aspect, the soy protein material may be a soy protein concentrate, which has a protein content of about 65% to about 90% on a moisture-free basis. Examples of suitable soy protein concentrates useful in the invention include Procon®, Alpha® DSP-C, Alpha® 12 and Alpha® 5800, which are commercially available from Solae, LLC (St. Louis, Mo.). Alternatively, soy protein concentrate may be blended with the soy protein isolate to substitute for a portion of the soy protein isolate as a source of soy protein material. Typically, if a soy protein concentrate is substituted for a portion of the soy protein isolate, the soy protein concentrate is substituted for up to about 40% of the soy protein isolate by weight, at most, and more preferably is substituted for up to about 30% of the soy protein isolate by weight.

In yet another aspect, the soy protein material may be soy flour, which has a protein content of about 49% to about 65% on a moisture-free basis. Alternatively, soy flour may be blended with soy protein isolate or soy protein concentrate.

In an alternate aspect, the soy protein material may be soy storage proteins that have been separated into three major fractions (11S, 7S, and 2S) on the basis of sedimentation in a centrifuge. In general, the 11S fraction is highly enriched in glycinins, and the 7S fraction is highly enriched in beta-conglycinins. In still yet another aspect, the soy protein material may be a high oleic soy protein.

In general, the starting soy protein material typically comprises a mixture of proteins having a range of sizes. In one aspect, the proteins in the soy material may range in size from about 1000 Daltons to about 500,000 Daltons. In another aspect, the proteins in the soy material may range in size from about 5000 Daltons to about 100,000 Daltons.

### Protein Hydrolysate

In one aspect of the present invention, the invention relates to a protein hydrolysate obtainable by the process according to the invention.

In a protein hydrolysate according to this aspect of the invention, the hydrolyzed protein material comprises a reduced content of polypeptide fragments having C-terminal hydrophobic amino acid residues. A protein hydrolysate according to this aspect of the invention has a superior taste and is less bitter than previously known protein hydrolysates having a similar degree of hydrolysis.

The degree of hydrolysis is known to influence the degree of bitterness of hydrolyzed protein, a high degree of hydrolysis generally leading to more bitterness. Further an increase in degree of hydrolysis may change the functionality of the protein material, as the solubility and/or suspendability increases. Typically, as the degree of hydrolysis increases from about 1% to about 20%, the protein hydrolysate has increased transparency, increased bitterness and, in relation to soy protein material, a decreased grain sensory attribute. Stated another way, as the degree of hydrolysis decreases from about 20% to about 1%, the protein hydrolysate has decreased transparency, decreased bitterness and, in relation to soy protein material, an increased grain sensory attribute.

It is desired to have a high degree of hydrolysis in certain food product as a higher degree of hydrolysis improves the functionality of the protein material. Especially when used in beverages the degree of hydrolysis is preferably high, i.e. around 10-20%.

Accordingly, a protein hydrolysate produced according to the invention preferably has a degree of hydrolysis from about 1% to about 20%, more preferably from about 5% to about 15%.

The protein hydrolysate, compared with the protein starting material, will comprise a mixture of polypeptide fragments of varying length and molecular weights. In general the polypeptide fragments will have less hydrophobic amino acid residues at the C-terminus.

### Food Products Comprising the Protein Hydrolysate

In one aspect of the invention, the processes according to the invention are used to produce or prepare a food product. In one particular aspect, the processes according to the invention are used for reducing the bitterness of a protein hydrolysate (e.g. to add to a food product) or they are used for reducing the bitterness of a food product.

In a further aspect, the present invention provides a food product comprising an edible material and any of the protein hydrolysates described herein.

The selection of the particular protein hydrolysate to combine with the edible material depends on the food product to be produced.

Several edible materials are suitable for use in the invention. The edible material may be a liquid or a solid. In one preferred aspect, the edible material is a liquid and is utilized to make a food product that is a liquid beverage. When the food product is a beverage, typically it is desirable to utilize a substantially transparent protein hydrolysate, such as a protein hydrolysate having a degree of hydrolysis closer to 20% than 1%.

Non-limiting examples of protein fortified liquid beverages suitable for inclusion of a protein hydrolysate according to the invention include carbonated fortified soft drinks, beer, fruit juices, fruit drinks, fruit-flavored drinks, vegetable drinks, nutritional drinks, energy drinks, sports drinks, soy milk drinks, flavored soy drinks, rice milk drinks, infant formula, enteral formulae, dry blended beverages, coffee drinks, or combinations thereof.

In another aspect, the food product may be a dairy product. The dairy product may be a liquid beverage such as milk, flavored milk drinks, goat milk, liquid yogurt, or buttermilk. Other suitable examples of dairy products include, but are not limited to, cheese, ice cream, ice cream products, yogurt, whipping cream, sour cream, or cottage cheese.

In an alternate aspect, the food product may be a nutritional supplement. The nutritional supplement may be liquid or solid. In another alternate aspect, the food product may be food bar, such as a granola bar, a cereal bar, a nutrition bar or an energy bar, or it may be soy protein nuggets.

In still another aspect, the food product may be a cereal-based product, such as a breakfast cereal product or a pasta product.

In still another aspect, the food product may be an extrudate, such as nuggets, crisps, puffs, or a pasta product.

In yet another aspect, the food product may be a meat product or a meat analogue product. Examples of meat products include, but are not limited to, processed meat, comminuted meat, and whole muscle meat. The meat may be animal meat or seafood meat. The meat analogue may be a textured vegetable or dairy protein that mimics animal or seafood meat in texture. The meat analogue may be part or all of the meat in a food product.

In another aspect of the invention the peptidases or the peptidase preparations according to the invention are added “in situ” to the food product to be treated. For example in the preparation of beverages, the enzymes or enzyme preparations could be added to the product with all other ingredients and perform the desired activities “in situ”. Similar “in situ”-applications for other food products, e.g. cheese, are also feasible.

### EXAMPLES

### Example 1

### Construction of a Recombinant Expression Strain for the Aspergillus Oryzae Carboxypeptidase

To clone the Aspergillus oryzae carboxypeptidase Y (CPY), the sequence of the predicted protein Q2TYA1 from the UniProt database (The Universal Protein Resource (UniProt) Nucleic Acids Res. 36:D190-D195 (2008)) was used to identify the corresponding coding sequence in the published A. oryzae genome sequence (Nature 438:1157-1161 (2005)). The CPY gene, thus identified, was expressed recombinantly in Aspergillus oryzae using standard techniques (Christensen, T. et al., (1988), Biotechnology 6, 1419-1422; WO 04/032648) in order to obtain CPY protein for testing. Briefly, the CPY gene was amplified from A. oryzae genomic DNA with standard PCR techniques using the following primers:

| | |
|---|---|
| 611: | TAGGGATCCTCACCATGAGAGTATTGCCGGCTACTTTGCTG |
| 617: | GAACCATTCACCACCCAACCAG |

The PCR product was restricted with BamHI and cloned into the BamHI and NruI sites of the Aspergillus expression vector pMStr100. pMStr100 was made from pMStr57 (WO 04/032648) by adding an in-frame tag with the sequence RHQHQHQH, by cloning the following DNA fragment into the NruI and XhoI sites:

TCGCGACATCAGCACCAGCATCAGCACTGACTCGAG
AGCGCTGTAGTCGTGGTCGTAGTCGTGACTGAGCTC

The pMStr100-based construct, pMStr125, was transformed into Aspergillus oryzae strain BECh2 (WO 00/39322).

### Example 2

### Recombinant Expression of the A. Oryzae CPY Protein

An Aspergillus oryzae transformant constructed as described above was fermented in 150 ml DAP2C-1 medium (WO 04/032648), with 2% glucose substituted for maltodextrin, in 500 ml fluted shake flasks incubated at 30° C. in a shaking platform incubator rotating at 200 RPM for 3 days.

### Example 3

### Purification and Characterization of the A. Oryzae CPY Protein

End-point carboxypeptidase assays using Z-Ala-Xxx-OH substrates were used as purification assay and for characterisation of the enzyme. ‘Z’ is an abbreviation for benzyloxycarbonyl and Xxx is the three letter abbreviation for an amino acid residue.

A fermented culture broth of Example 2 was used as start material for the purification. The culture broth was filtered though filtration cloth to remove the majority of the fungal hyphae. The filtrate was then 0.2 micrometer filtrated though a filtration unit from Nalgene in order to remove the rest of the Aspergillus host. Solid ammonium sulphate was added to the 0.2 micrometer filtrate to give a 2M final (NH₄)₂SO₄ concentration and pH was adjusted to pH 6. The solution was mixed gently with a magnetic stirrer during the (NH₄)₂SO₄ addition and the stirring was continued for 30 minutes after the addition to bring the system in equilibrium. Then the solution was applied to a Phenyl Toyopearl column equilibrated in 10 mM succinic acid/NaOH, 2M (NH₄)₂SO₄, pH 6.0. After washing the column with several column volumes of the equilibration buffer, the column was eluted with a linear gradient over ten column volumes between the equilibration buffer and 10 mM succinic acid/NaOH, pH 6.0.

Fractions, collected during elution, were tested for carboxypeptidase Y activity (using the Purification assay as described below). However, it turned out that the carboxypeptidase was not yet eluted from the column. Therefore the column was eluted with 10 mM succinic acid/NaOH, pH 6.0 with 25% (v/v) 2-pronanol. Fractions collected during this elution contained the carboxypeptidase Y activity. The eluted enzyme (in 25% (v/v) 2-propanol) was quickly transferred to 10 mM MES/NaOH, 0.5 mM CaCl₂, pH 6.0 on a G25 sephadex column and applied to a Q-sepharose FF column equilibrated in 20 mM MES/NaOH, 0.5 mM CaCl₂, pH 6.0. After washing the column with several column volumes of the equilibration buffer, the peptidase was eluted over five column volumes with a linear NaCl gradient (0->0.5 M). Fractions, collected during elution, were tested for carboxypeptidase Y activity (using the Purification assay). Fractions with carboxypeptidase activity were analysed by SDS-PAGE. Fractions where only a double band was seen on the coomassie stained SDS-PAGE gel were pooled as the purified preparation which was used for further characterization.

### 1) Purification Assay:

100 microliter Z-Ala-Phe-OH substrate (Bachem C-1155) (50 mg dissolved in 1.0 ml DMSO and further diluted 25× in 0.01% Triton X-100) was mixed with 150 microliter Assay buffer (100 mM succinic acid, 100 mM HEPES, 100 mM CHES, 100 mM CABS, 1 mM CaCl₂, 150 mM KCl, 0.01% Triton X-100, pH 6.0) in an Eppendorf tube and placed on ice. 50 microliter peptidase sample (diluted in 0.01% Triton X-100) was added. The assay was initiated by transferring the Eppendorf tube to an Eppendorf thermomixer, which was set to 37° C. The tube was incubated for 15 minutes on the Eppendorf thermomixer at its highest shaking rate (1400 rpm.). The tube was then transferred back to the ice bath and when the tube had cooled, 500 microliter Stop reagent (17.9 g TCA+29.9 g Na-acetate trihydrate+19.0 ml conc. CH₃COOH and deionised water ad 500 ml) was added and the tube was vortexed and left for 15 min at room temperature (to ensure complete inactivation). The tube was centrifuged (15000×g, 3 min, room temp), 30 microliter supernatant was transferred to a microtiter plate and 225 microliter freshly prepared OPA-reagent (3.81 g disodium tetraborate and 1.00 g SDS was dissolved in approx. 80 ml deionised water—just before use 80 mg ortho-phtaldialdehyde dissolved in 2 ml ethanol was added and then 1.0 ml 10% (w/v) DTE and finally the volume was adjusted ad 100 ml with deionised water) was added. After 2 minutes, A₃₄₀ was read in a MTP reader. The A₃₄₀ measurement relative to an enzyme blind (50 microliter 0.01% Triton X-100 instead of peptidase sample) was a measure of carboxypeptidase activity.

### 2) Characterisation Assay:

The purification assay above was repeated using Z-Ala-Phe-OH (Bachem C-1155) as substrate. The pH-activity profile was performed at 37° C. in Assay buffer adjusted with HCl or NaOH to pH-values: 2.0, 3.0, 4.0, 5.0, 6.0, 7.0, 8.0, 9.0, 10.0, and 11.0. The Temperature-activity profile was performed at pH 6.0 and varying the temperature of the assay. Results are shown in Tables 1 and 2 below.

TABLE 1
pH-activity profile

| | CPY from |
|---|---|
| pH | Aspergillus oryzae |
| 2 | 0.03 |
| 3 | 0.06 |
| 4 | 0.26 |
| 5 | 0.49 |
| 6 | 1.00 |
| 7 | 0.69 |
| 8 | 0.10 |
| 9 | 0.01 |
| 10 | 0.01 |
| 11 | 0.01 |

The reported activity data in Table 1 is relative to the optimal activity (at pH 6.0).

TABLE 2
Temperature activity profile at pH 6.0

| | CPY from |
|---|---|
| Temp (° C.) | Aspergillus oryzae |
| 15 | 0.11 |
| 25 | 0.32 |
| 37 | 0.59 |
| 50 | 1.00 |
| 60 | 0.33 |

The reported activity data in Table 2 is relative to the optimal activity (at 50° C.).

The purification assay above was used for obtaining the pH-stability profile: the residual activity after 2 hours at 37° C. For the pH-stability profile, the peptidase was diluted 10× in the assay buffers (pH 2 to pH 11) and incubated for 2 hours at 37° C. After incubation the peptidase samples were transferred to pH 6, before assay for residual activity, by dilution in the pH 6 Assay buffer. The results are shown in Table 3 below.

TABLE 3
pH-stability profile

| | CPY from |
|---|---|
| pH | Aspergillus oryzae |
| 2.0 | 0.00 |
| 3.0 | 0.00 |
| 4.0 | 0.68 |
| 5.0 | 0.94 |
| 6.0 | 0.97 |
| 7.0 | 0.74 |
| 8.0 | 0.13 |
| 9.0 | 0.05 |
| 10.0 | 0.01 |
| 11.0 | 0.01 |
| 6.0 and after 2 | 1.00 |
| hours at 5° C. | |

The reported activity data in Table 3 is residual activity after 2 hours at 37° C.

### Specificity on Seven Z-Ala-Xxx-OH Substrates:

The purification assay above was repeated using Z-Ala-Phe-OH (Bachem C-1155), Z-Ala-Ala-OH (Bachem C-1045), Z-Ala-Leu-OH (Bachem C-3155), Z-Ala-Pro-OH (Bachem C-1185), Z-Ala-Glu-OH (Bachem C-1075), Z-Ala-Lys-OH (Bachem C-1140), Z-Ala-His-OH (Bachem C-1120) as substrates at 37° C. and pH 6.0. The relative activity is a measure of the P1′ specificity for the carboxypeptidase. The result is shown in Table 4 below.

TABLE 4
P1′-specificity on Z-Ala-Xxx-OH at pH 6.0

| X | Xxx | |
|---|---|---|
| (1-letter | (3-letter | CPY from |
| abbreviation) | abbreviation) | Aspergillus oryzae |
| A | Ala | 0.33 |
| E | Glu | 0.00 |
| F | Phe | 0.96 |
| H | His | 0.06 |
| K | Lys | 0.00 |
| L | Leu | 1.00 |
| P | Pro | 0.00 |

The reported activity data in Table 4 is relative to the optimal activity (for Z-Ala-Leu-OH).

### Other Characteristics:

Carboxypeptidase Y from Aspergillus oryzae was found to be inhibited by 1,10-phenanthroline and PMSF.

The relative molecular weight determined by SDS-PAGE was:

Carboxypeptidase Y from Aspergillus oryzae: Mᵣ=approx. 52 kDa.

The N-terminal sequence was:

Carboxypeptidase Y from Aspergillus oryzae: KADPSALGID

The molecular weight determined from the translated DNA sequence and the N-terminal sequence:

Carboxypeptidase Y from Aspergillus oryzae: 46898 Da.

From these data, it is likely to assume that the carboxypeptidase Y enzyme is glycosylated.

### Example 4

### Sensory Evaluation Experiments for Carboxypeptidase Y from Aspergillus oryzae (CPY)

The purpose of this experiment was to analyse the bitterness of soy hydrolysed with an endoprotease alone and in combination with carboxypeptidase Y from Aspergillus oryzae obtained as described in Example 3. Three different endoproteases were tested: Alcalase, Savinase (both available from Novozymes NS, Denmark), SP1 (a protease derived from Nocardiopsis sp. NRRL 18262 disclosed in WO01/58276). The untreated substrate (SUPRO® 500E, available from Solae, LLC, St. Louis, Mo.) was used as the reference.

### Method:

Soy preparation: 15 g soy isolate (Supro 500E)+285 g water. The soy was added slowly to the Milli Q water (room temperature) on a magnetic stirrer. The rpm varied depending on characteristics of the soy (avoid foaming). The soy solution dispersed from 15 to 30 min. depending on whether all soy was suspended/solubilized.

pH was adjusted to 8.0 with 4 N NaOH.

Alcalase, Savinase or SP1 was dosed with 300 mg enzyme protein (ep)/kg soy. CPY was dosed with 100 mg enzyme protein (ep)/kg soy. Enzyme was stored on ice during handling.

### Enzyme Treatment:

The enzyme treatment with combinations of CPY and Alcalase or Savinase or SP1 was done in two steps due to difference in pH and temperature optimum.

Alcalase (300 mg/kg)+CPY (100 mg/kg) Treatment:

First Alcalase was added at pH8 and 60° C. and incubated for 1 h. Then pH was adjusted with HCl to pH6 and temperature was adjusted to 50° C. prior to the addition of CPY and the solution was incubated for another 1 h at 50° C.

Savinase (300 mg/kg)+CPY (100 mg/kg) Treatment:

First Savinase was added at pH8 and 60° C. and incubated for 1 h. Then pH was adjusted with HCl to pH6 and temperature was adjusted to 50° C. prior to the addition of CPY and the solution was incubated for another 1 h at 50° C.

SP1 (300 mg/kg)+CPY (100 mg/kg) Treatment:

First SP1 was added at pH8 and 60° C. and incubated for 1 h. Then pH was adjusted with HCl to pH6 and temperature was adjusted to 50° C. prior to the addition of CPY and the solution was incubated for another 1 h at 50° C.

### Heat Inactivation/Storage:

Immediately after enzyme treatment, the samples were heat treated 15 min. a 85° C. in a shaking water bath. The samples were cooled on ice and refrigerated at 4° C. over night.

### Degree of Hydrolysis:

Degree of hydrolysis of the suspension was measured using the OPA (o-phthatdialdehyde) assay 20 microliter sample diluted with MilliQ water (normally 10-80×) was mixed with 180 microliter OPA reagent (4 mM disodium tetraborate, 0.1% SDS, 0.24 mM OPA, 0.24 mM DTT) in the well of a microtiter plate. Absorbance at 340 nm was measured. A standard curve with L-serine was included (0-0.5 mg/ml). In the table below, results are reported as increase in degree of hydrolysis relative to starting material Supro 500E.

### Sensory Evaluation:

Samples were equilibrated to room temperature before serving. The flasks with samples were shaken well before pouring the slurry. The Solae Qualitative Screening (SQS) method was used for sensory evaluation (PCT/US08/60486). The panel consisted of 5-10 assessors.

### Score on Bitterness:

+1 slightly more bitter than reference
+2 moderately more bitter than reference
+3 extremely more bitter than reference
0 like reference
−1 slightly less bitter than reference
−2 moderately less bitter than reference
−3 extremely less bitter than reference

### Results:

Taste evaluation for bitterness of soy treated with combinations of endoproteases and exopeptidase was performed. The results are shown as an average of all panelist's scores. CPY removes the bitterness from soy treated with 3 different endoproteases. A big effect was seen of CPY on SP1 and Alcalase treated soy, while a smaller effect was seen on Savinase treated soy. It is clear that CPY does remove or reduce bitterness of soy hydrolysed with several different endoproteases.

The results of the taste evaluation of the enzyme treated samples are shown in Table 5, and the degree of hydrolysis of the same samples is shown in Table 6.

TABLE 5
Taste evaluation results

| Enzyme | Bitterness Score |
|---|---|
| treatment | of hydrolysed soy |
| SP1 | 1.6 |
| SP1/CPY | 0.0 |
| Alcalase | 1.8 |
| Alcalase/CPY | 0.3 |
| Savinase | 2.0 |
| Savinase/CPY | 1.0 |

TABLE 6
Degree of hydrolysis (results are reported as increase in degree
of hydrolysis relative to starting material Supro 500E):

| | | SP1 + | | Alcalase + | | Savinase + |
|---|---|---|---|---|---|---|
| | SP1 | CPY | Alcalase | CPY | Savinase | CPY |
| DH | 10 ± 1 | 11 ± 1 | 10 ± 1 | 11 ± 2 | 11 ± 1 | 12 ± 1 |
| (%) | | | | | | |

### Example 5

### Sensory Evaluation Experiments for Carboxypeptidase Y from Aspergillus Oryzae (CPY) and Another Carboxypeptidase

The purpose of this experiment was to analyse the effect of CPD (SEQ ID NO: 2 in WO9814599-A1) and CPY (obtained as described in example 3) for bitterness reduction of soy-hydrolysates made with the endoprotease Alcalase (available from Novozymes NS, Denmark). The untreated soy substrate (SUPRO® 500E, available from Solae, LLC, St. Louis, Mo.) was used as the reference. CPD is carboxypeptidase D from Aspergillus oryzae with preference for basic amino acids (lysine and arginine). CPY is carboxypeptidase Y from Aspergillus oryzae with preference for hydrophobic amino acids

### Method:

Soy preparation: 15 g soy (Supro 500E)+285 g water. The soy was added slowly to the Milli Q water (room temperature) on a magnetic stirrer. The rpm varied depending on characteristics of the soy (avoid foaming). The soy solution was dispersed from 15 to 30 min. depending on whether all soy was suspended/solubilized.

### pH Adjustment:

pH was adjusted to 8.0 with 4 N NaOH.

The enzyme treatment with combinations of CPY or CPD and Alcalase was done in two steps due to difference in pH and temperature optimum.

Alcalase (300 mg/kg)+CPD (100 mg/kg) or CPY (100 mg/kg) Treatment:

First Alcalase was added at pH8 and 60° C. and incubated for 1 h. Then pH was adjusted with HCl to pH6 and temperature was adjusted 50° C. prior to the addition of CPD or CPY and the solution was incubated for another 1 h at 50° C.

### Heat Inactivation/Storage:

Immediately after enzyme treatment, the samples were heat treated 15 min. a 85° C. in a shaking water bath. Samples were cooled on ice and refrigerated at 4° C. over night.

### Degree of Hydrolysis:

Degree of hydrolysis of the suspension was measured using the OPA (o-phthatdialdehyde) assay 20 microliter sample diluted with MilliQ water (normally 10-80×) was mixed with 180 microliter OPA reagent (4 mM disodium tetraborate, 0.1% SDS, 0.24 mM OPA, 0.24 mM DTT) in the well of a microtiter plate. Absorbance at 340 nm was measured. A standard curve with L-serine was included (0-0.5 mg/ml). In the table below, results are reported as increase in degree of hydrolysis relative to starting material Supro 500E.

### Sensory Evaluation:

Samples were equilibrated to room temperature before serving. The flasks with samples were shaken well before pouring the slurry. The Solae Qualitative Screening (SQS) method was used for sensory evaluation (PCT/US08/60486). The panel consisted of 5-10 assessors.

### Score on Bitterness:

+1 slightly more bitter than reference
+2 moderately more bitter than reference
+3 extremely more bitter than reference
0 like reference
−1 slightly less bitter than reference
−2 moderately less bitter than reference
−3 extremely less bitter than reference

### Results:

Taste evaluation for bitterness of soy treated with combinations of the endoprotease Alcalase and the two carboxypeptidases CPD and CPY was performed. The results are shown as an average of all panelist's scores. The results show that CPY removed bitterness more efficiently than CPD.

The results of the taste evaluation of the enzyme treated samples are shown in Table 7, and the degree of hydrolysis of the same samples is shown in Table 8.

TABLE 7
Taste evaluation results

| | Bitterness Score of |
|---|---|
| Enzyme treatment | hydrolysed soy |
| Alcalase | 2.0 |
| Alcalase + CPD | 0.6 |
| Alcalase + CPY | −0.4 |

TABLE 8
Degree of hydrolysis (results are reported as increase in degree
of hydrolysis relative to starting material Supro 500E):

| | | Alcalcase + | Alcalase + |
|---|---|---|---|
| | Alcalase | CPD | CPY |
| DH (%) | 10 ± 1 | 11 ± 1 | 10 ± 1 |

### Example 6

### Hydrolysates Prepared with SP1 or ALCALASE® with or without Carboxypeptidase Y from Aspergillus oryzae (CPY)

Isolated soy protein was hydrolyzed with SP1 (a protease derived from Nocardiopsis sp. NRRL 18262 disclosed in WO01/58276) or ALCALASE® (available from Novozymes NS, Denmark) alone or in combination with varying amounts of CPY. SP1 was used at 1500 mg/kg soy, Alcalase was used at 5.0% curd solid basis, and CPY was used at 0, 10, 25, or 50 mg/kg soy. The endopeptidase reactions were performed at pH 8 and 60° C. for 60 min; the exopeptidase reactions were performed at pH 6.0 and 50° C. for 60 min.

The degree of hydrolysis of the soluble fraction was determined using the trinitrobenzenesulfonic acid (TNBS) method described in Adler-Nissen, 1979, J. Agric. Food Chem. 27(6):1256-1262. Reaction of primary amino groups with TNBS forms trinitrobenzene amines, which are visible chromophores that can be detected at 416 nm. The sensory profiles of these hydrolysates were analyzed using the SQS method as detailed above. The soluble fraction of each hydrolysate was compared to the soluble fraction of the appropriate endopeptidase-treated hydrolysate.

Table 9 below presents the degree of hydrolysis of the soluble fraction of each hydrolysate. Higher concentrations of CPY led to higher degrees of hydrolysis. Table 9 also presents the sensory diagnostic scores of the soluble fractions presented as a slurry at neutral pH. The hydrolysates prepared with the highest concentration of CPY at 50 mg CPY/kg soy in combination with an endopeptidase had slightly decreased bitterness scores relative to the controls with either SP1 or Alcalase hydrolysates.

TABLE 9
Sensory Analysis of Hydrolysates at Neutral pH.

| | Degree of | Bitterness |
|---|---|---|
| Enzyme Treatment | Hydrolysis (%) | Score |
| SP1 (Control) | 15.9 | 0 |
| SP1 + 50 mg CPY | 18.2 | 0.5 |
| Alcalase (Control) | 21.1 | 0 |
| Alcalase + 50 mg CPY | 23.4 | 0.5 |

The free amino acids of hydrolysates prepared with SP1+CPY at different doses were measured as follows:

A portion of sample was mixed with hydrochloric acid solution in a modified Kjeldahl flask. To prevent oxidation of the amino acids, as much oxygen as possible was removed from the flask by repeated freezing and thawing under vacuum. The neck of the flask was heat-sealed and the flask was heated in a 110° C. oven for 20 hours. Protein in the sample was hydrolyzed to amino acids by the hot hydrochloric acid solution. The samples were cooled, opened, mixed with internal standard, and adjusted to pH 2.2. The amino acids were separated on an ion exchange column by a pH gradient elution with controlled column temperatures. The separated amino acids were subsequently reacted with ninhydrin, forming color complex solutions that were measured spectrophotometrically. The concentration of each amino acid was quantitated against a standard solution of amino acids of known concentration and containing an internal standard, which was also injected into the amino acid analyzer. (REFERENCES: 1: Official Methods of Analysis of the AOAC International, 16th Edition, Method 994.12, Alternative III, Locator 4.1.11. 2: Methods in Enzymology, 1963, Vol. VI, pp. 819-831, “Chromatographic Determination of Amino Acids by the Use of Automatic Recording Equipment,” S. Moore and W. H. Stein. 3: J. Biol. Chem., 1954, Vol. 211, pp. 893-907, “Procedures for the Chromatographic Determination of Amino Acids on Four Percent Cross-Linked Sulfonated Polystyrene Resins,” S. Moore and W. H. Stein. 4: Amino Acid Determination—Methods and Techniques, 1978, 2nd Edition, S. Blackburn. 5: Research Project AR-108, 1982, “Vacuum Hydrolysis of Isolated Soy Proteins for the Determination of Amino Acid Profiles,” Ralston Purina Company. 6: Research Project CRX-4, 1982, “Amino Acid Profiles of Feed Ingredients using High Vacuum Hydrolysis,” Ralston Purina Company.)

The SP1 and SP1+CPY soluble fractions revealed that CPY cleaved alanine, arginine, glutamate, isoleucine, leucine, lysine, methionine, phenylalanine, tyrosine, and valine residues from the carboxyl terminal end of the fragments. In this experiment, the number of free amino acid residues released in SP1 soluble fractions was similar between the two lower concentrations of CPY, but treatment with the high concentration (50 mg) of CPY increased the number of released alanine, isoleucine, leucine, phenylalanine, tyrosine, and valine residues by at least two-fold (see Table 10 below).

TABLE 10
Free amino acid (moles/kg)

| | SP1 + 10 mg | SP1 + 25 mg | SP1 + 50 mg |
|---|---|---|---|
| | CPY/kg | CPY/kg | CPY/kg |
| Glu | 0.41 | 0.41 | 0.54 |
| Pro | 0 | 0 | 0.7 |
| Ala | 0.79 | 0.79 | 1.8 |
| Val | 1.54 | 1.37 | 4.36 |
| Met | 0.81 | 0.81 | 1.21 |
| Ile | 0.53 | 0.46 | 1.37 |
| Leu | 2.75 | 2.52 | 5.73 |
| Tyr | 0.88 | 0.77 | 1.82 |
| Phe | 1.39 | 1.27 | 3.09 |
| Lys | 0.89 | 0.89 | 1.16 |
| Arg | 0.34 | 0.29 | 0.52 |

1. A process for preparing a protein hydrolysate from a protein containing material comprising the steps of
a. hydrolyzing the protein containing material,
b. contacting the hydrolysed protein material with a carboxypeptidase comprising an amino acid sequence which has at least 70% identity to the amino acid sequence of SEQ ID NO:1 or amino acids 124-542 thereof.

2. The process according to claim 1, where the carboxypeptidase comprises an amino acid sequence which is identical to the amino acid sequence of SEQ ID NO:1 or amino acids 124-542 thereof.

3. The process according to claim 1, wherein the carboxypeptidase is a carboxypeptidase C.

4. The process according to claim 1, wherein the carboxypeptidase is a carboxypeptidase Y.

5. The process according to claim 1, wherein the protein containing material is hydrolyzed by means of an endoprotease.

6. The process according to claim 5, wherein the endoprotease is an endoprotease which preferentially produces C-terminal hydrophobic amino acid residues.

7. The process according to claim 1, wherein the protein containing material is selected from the group consisting of animal protein containing materials, vegetable protein containing materials, yeast protein containing materials or derivatives and combinations thereof.

8. The process according to claim 1, wherein the protein containing material is selected from the group consisting of milk protein containing materials, soy protein containing materials, yeast protein containing materials or derivatives and combinations thereof.

9. The process according to claim 1, wherein the protein containing material is a soy protein containing material.

10. The process according to claim 1, wherein the protein hydrolysate has a degree of hydrolysis from above 0% to about 30%.

11. The process according to claim 1, wherein the process is for reducing the bitterness of the protein hydrolysate.

12. A protein hydrolysate obtainable by the process according to claim 1.

13. A food product comprising a protein hydrolysate as defined in claim 12.

14. A food product according to claim 13, wherein the food product is a dry blended or liquid beverage.

15. A food product according to claim 13, wherein the food product is selected from the group consisting of nutritional supplements, clinical nutrition products, infant formulae, baby foods, extrudates, food bars, meat and meat analogue products, dairy products and breakfast cereals.


SEQUENCE LISTING (6 sequences)

SEQ ID NO: 1 - protein, 542 aa, Aspergillus oryzae
MRVLPATLLVGAASAAVPPLQQVLGRPEEGMSFSKPLHAFQEQLKTLSEDARKLWDEVANYFPDSMDHSPIFSLPKKHTRRPDSHWDHIVRGSDVQKIWVNNADGEKEREIDGKLEAYDLRIKKADPSALGIDPNVKQYTGYLDDNGNDKHLFYWFFESRNDPKNDPVVLWLNGGPGCSSLTGLFMELGPSSIDENIKPVYNDFSWNSNASVIFLDQPVNVGYSYSGSAVSDTVAAGKDVYALLSLFFKQFPEYAEQDFHIAGESYAGHYIPVFASEILAHKNRNINLKSVLIGNGLTDGLTQYGYYRPMGCGEGGYKAVLDEATCESMDNALPRCRSMIESCYNSESAWVCVPASIYCNNALIGPYQRTGQNVYDVRSKCEDESNLCYKGMGYVSEYLNKAEVREAVGAEVGGYDSCNFDINRNFLFHGDWMKPYHRLVPGLLEQIPVLIYAGDADYICNWLGNKAWTEALEWPGQKEYASAELEDLKIEQNEHTGKKIGQVKSHGNFTFMRLYGGGHMVPMDQPEASLEFFNRWLGGEWF

SEQ ID NO: 2 - protein, 419 aa, Aspergillus oryzae
KADPSALGIDPNVKQYTGYLDDNGNDKHLFYWFFESRNDPKNDPVVLWLNGGPGCSSLTGLFMELGPSSIDENIKPVYNDFSWNSNASVIFLDQPVNVGYSYSGSAVSDTVAAGKDVYALLSLFFKQFPEYAEQDFHIAGESYAGHYIPVFASEILAHKNRNINLKSVLIGNGLTDGLTQYGYYRPMGCGEGGYKAVLDEATCESMDNALPRCRSMIESCYNSESAWVCVPASIYCNNALIGPYQRTGQNVYDVRSKCEDESNLCYKGMGYVSEYLNKAEVREAVGAEVGGYDSCNFDINRNFLFHGDWMKPYHRLVPGLLEQIPVLIYAGDADYICNWLGNKAWTEALEWPGQKEYASAELEDLKIEQNEHTGKKIGQVKSHGNFTFMRLYGGGHMVPMDQPEASLEFFNRWLGGEWF

SEQ ID NO: 3 - protein, 10 aa, Aspergillus oryzae
KADPSALGID

SEQ ID NO: 4 - DNA, 41 bp, Artificial sequence
  Primer 611
tagggatcctcaccatgagagtattgccggctactttgctg

SEQ ID NO: 5 - DNA, 22 bp, Artificial sequence
  Primer 617
gaaccattcaccacccaaccag

SEQ ID NO: 6 - DNA, 36 bp, Artificial sequence
  HQ-tag
tcgcgacatcagcaccagcatcagcactgactcgag